(19) United States
(12) Patent Application Publication
(10) Pub. No.: US 2019/0290718 A1
(43) Pub. Date: Sep. 26, 2019

(54) COMPOSITION COMPRISING BORTEZOMIB

(71) Applicant: FTF Pharma Private Limited, Ahmedabad (IN)

(72) Inventors: Jayanta Kumar Mandal, Ahmedabad (IN); Sandip P. Mehta, Ahmedabad (IN)

(73) Assignee: FTF Pharma Private Limited, Ahmedabad (IN)

(21) Appl. No.: 16/435,734

(22) Filed: Jun. 10, 2019

Related U.S. Application Data
(63) Continuation-in-part of application No. 15/230,310, filed on Aug. 5, 2016, now Pat. No. 10,314,880.

(30) Foreign Application Priority Data
Aug. 6, 2015 (IN) 2968/MUM/2015

(51) Int. Cl. A61K 38/05; A61K 47/02; A61K 9/00; A61K 9/08

(52) U.S. Cl. CPC A61K 38/05; A61K 9/08; A61K 9/0019; A61K 47/02

(57) ABSTRACT

The present invention provides a pharmaceutical composition of bortezomib. In particular, present invention provides ready-to-dilute injectable formulations comprising bortezomib, a non-aqueous solvent and a pH modifying or pH adjusting agent. Further, the present invention also relates to the process of manufacturing ready-to-dilute injectable formulations comprising bortezomib. The product prepared by following the process disclosed in the present application is stable at room temperature and possesses adequate shelf life (for example for 18-24 months).

### CROSS-REFERENCE TO RELATED APPLICATIONS

This application is a continuation-in-part application of U.S. patent application Ser. No. 15/230,310 filed on 5 Aug. 2016, which is incorporated herein by reference in its entirety.

### FIELD OF THE INVENTION

The present invention relates to a pharmaceutical composition comprising bortezomib. In particular, present invention relates to the ready-to-dilute injectable formulations comprising bortezomib, a non-aqueous solvent, a pH modifying or pH adjusting agent and optionally an anti-oxidant. Further, the present invention also relates to the process of manufacturing ready-to-dilute injectable formulations comprising bortezomib. The product prepared using the process of the invention is stable at room temperature and possesses adequate shelf life (for example for 18-24 months).

### BACKGROUND OF THE INVENTION

The high price of many innovative drugs, which is in part due to costs, time and risks involved in drug development, calls for more efficient approaches to bring drugs to the market. Translational research has been identified as an important component of such strategies. Translational research is not only a function of quality science but also the collaboration of academia and industry, which is best exemplified by success history of bortezomib.

Dipeptide boronate named MG-341 was designed by a straightforward medicinal chemistry approach. Bortezomib was first described in U.S. Pat. No. 5,780,454 with the code MG-341. Because of being proteosome inhibitor, bortezomib is involved in inflammatory responses via activation of NF-kB.

WO 99015183 discloses a method of treatment of inflammatory and autoimmune diseases by administering proteosome inhibitors.

Bortezomib worked very well in animal models of inflammation, especially in rheumatoid arthritis after phase I trial. After phase II trial, multiple myeloma found to be more susceptible to bortezomib based on the study of bortezomib in which bortezomib removed all signs of cancer in a patient of advanced stages of multiple myeloma. Thus, bortezomib was approved by FDA as an injectable small molecule for the treatment of multiple myeloma.

Bortezomib, [(1R)-3-methyl-1-[[(2S)-3-phenyl-2-(pyrazine-2-carbonylamino) propanoyl] amino] butyl] boronic acid, is the first therapeutic anti-neoplastic proteosome inhibitor. Bortezomib is a modified dipeptidyl boronic acid derived from leucine and phenylalanine. Bortezomib works via inhibition of proteolytic activity of proteosome and thus it inhibits degradation of polyubiquitinated proteins responsible for catalysis of proteins.

Bortezomib is isolated as trimeric boroxine. Bortezomib as such has poor water solubility, so to overcome this difficulty various dosage forms are formulated.

U.S. Pat. Nos. 6,958,319 and 6,713,446 disclose stable pharmaceutical compositions of boronic acid compounds which are prepared by lyophilization of an aqueous mixture comprising a boronic acid compound and a moiety derived from sugar produces a stable composition that readily releases the boronic acid compound upon dissolution in aqueous media.

U.S. Pat. No. 6,699,835 discloses lyophilized powder form of bortezomib with D-mannitol.

US Patent Application Publication No. 2011/0178470 discloses oral and parenteral formulations of bortezomib or its pharmaceutically acceptable salts or solvates, in the form of ready-to-use solution, lyophilized forms or physical admixtures. This patent further discloses processes for preparation of these compositions and methods of using compositions for treating various types of cancers in mammals.

US Patent Application Publication No. 2012/0172808 discloses storage-stable liquid pharmaceutical composition that includes bortezomib in a therapeutically effective amount, the composition comprising a single-phase liquid formulation comprising a substantially non-aqueous solvent system suitable for injection, an aqueous buffer, and bortezomib wherein the solvent system comprises a predominant component propylene glycol.

PCT Patent Application publication number WO 2010/089768 discloses a bortezomib formulation in which bortezomib is lyophilized with tromethamine.

PCT Patent Application publication number WO 2010/0114982 discloses lyophilized cake formulation of bortezomib containing bortezomib, cyclodextrin, bulking agent and surfactant.

US patent application publication number 2011/0230441 discloses multi-dose formulation of bortezomib with improved stability, wherein bortezomib is in liquid form suitable for injection with propylene glycol solvent.

US patent application publication number 2012/0083457 discloses lyophilized composition of bortezomib and boric acid in a mass ratio of boric acid to bortezomib is from 1:1 to 10:1.

EP 2644189 discloses storage-stable multi-dose liquid formulation of bortezomib with improved stability.

Existed lyophilized products have several disadvantages which includes time consuming reconstitution process, maintenance of dose precision, limited stability in solution form and also lyophilization process for cytotoxic drugs requires exposure of healthcare professional to cytotoxic vapor of the drugs.

To overcome above mentioned problems associated with lyophilized formulation and also to enhance stability and bioavailability of active ingredient, present invention provides ready-to-dilute injectable pharmaceutical formulations of bortezomib with significantly improved solubility, stability/comparable stability which do not contain buffers for maintaining pH of the formulations.

Further, manufacturing process of bortezomib solution is critical and many parameters need to be checked and taken care of at each and every stage in order to protect formation of impurities/related substances/degradants and maintain the purity of the drug product. The present invention, therefore, also provides the manufacturing process for preparing bortezomib solution which provides ready-to-dilute bortezomib formulation having room temperature stability and adequate shelf life, for example for 18-24 months.

One specific aspect of the invention provides a composition for a ready-to-dilute injectable pharmaceutical formulation of bortezomib. Typically, this composition is in a solution form. In particular, compositions of the invention comprises:
about 7.0 mg/mL to about 17.5 mg/mL bortezomib;
a non-aqueous solvent;
a non-buffer pH modifying or pH adjusting agent; and
optionally an anti-oxidant,
   wherein the amount of dissolved oxygen in said composition is 10 parts per million (ppm) or less.

In some embodiments, the non-aqueous solvent is selected from the group consisting of a fixed oil, an alcohol, glycerin, polyethylene glycol, propylene glycol, monothioglycerol, dimethylsulfoxide, ethyl ether, liquid paraffin, and a combination thereof.

Yet in other embodiments, the non-buffer pH modifying or pH adjusting agent is selected from the group consisting of hydrochloric acid, maleic acid, ascorbic acid, oxalic acid, carbonic acid, and a combination thereof. In one particular embodiment, the pH modifying or pH adjusting agent comprises hydrochloric acid.

Still in other embodiments, when the anti-oxidant is present, typically the antioxidant is selected from the group consisting of d-α-tocopheryl polyethylene glycol 1000 succinate (“Vitamin-E TPGS”), monothioglycerol, L-cysteine hydrochloride, sodium sulphite, L-methionine, disodium EDTA, and a combination thereof.

In other embodiments, pH of the composition is between about pH 1.0 and about pH 7.0.

In a further embodiment of the invention, the amount of total impurities (i.e., all the impurities combined) present in said composition is not more than about 10.0% w/w.

In some embodiments, the composition is stable at room temperature.

Yet in other embodiments, the composition upon dilution with pharmaceutically acceptable diluent is stable from about 8 hours up to about 7 days.

Another aspect of the invention provides a non-aqueous solution composition of bortezomib produced by the following process:
removing dissolved oxygen from a non-aqueous solvent to produce a substantially oxygen free non-aqueous solvent;
adding a pH modifying or pH adjusting agent to adjust the pH of said substantially oxygen free non-aqueous solvent to from about pH 1.0 to about pH 7.0;
adding bortezomib to said pH adjusted substantially oxygen free non-aqueous solvent to produce said non-aqueous solution composition, wherein said non-aqueous solution composition comprises from about 7.0 mg/mL to about 17.5 mg/mL of bortezomib; and
optionally adding an anti-oxidant to said non-aqueous solution composition.

In some embodiments, the process is carried out at temperature of about 20° C. to about 30° C. and under relative humidity (RH) of not more than about 55% RH.

Typically, the process is carried out in the absence of visible light.

Still in other embodiments, the process of removing dissolved oxygen comprises purging said non-aqueous solvent with nitrogen to remove dissolved oxygen.

Yet in other embodiments, the process further comprises a step of filtering said non-aqueous solution composition under nitrogen atmosphere to maintain head space oxygen of not more than 15% v/v. In some instances, the process further comprises a step of filling vials with said filtered non-aqueous solution composition under nitrogen atmosphere in the absence of visible light.

Still in other embodiments, the process comprises use of a container in which occupied volume of said container by the batch is not less than 20% v/v of the rated volume of said container.

In other embodiments, the amount of dissolved oxygen level is maintained throughout the said process at not more than 10 parts per million (ppm).

Still another aspect of the invention provides a for producing a non-aqueous solution composition of bortezomib disclosed herein. The process includes:
removing dissolved oxygen from a non-aqueous solvent to produce a substantially oxygen free non-aqueous solvent;
adding a pH modifying or pH adjusting agent to adjust the pH of said substantially oxygen free non-aqueous solvent to from about pH 1.0 to about pH 7.0;
adding bortezomib to said pH adjusted substantially oxygen free non-aqueous solvent to produce said non-aqueous solution composition of claim 1; and
optionally adding an anti-oxidant to said non-aqueous solution composition.

In some embodiments, the dissolved oxygen is removed such that the level of dissolved oxygen is not more than 10 parts per million (ppm). This level of dissolved oxygen (i.e., not more than 10 ppm) is then maintained throughout the process.

Still in other embodiments, the step of removing dissolved oxygen from said non-aqueous solvent comprises purging with nitrogen gas.

### SUMMARY OF THE INVENTION

It has been found that the solubility of proteosome inhibitors, such as bortezomib, is significantly enhanced when it is formulated with non-aqueous solvents.

The present invention relates to the ready-to-dilute injectable formulations of bortezomib with increased stability. In one embodiment, present invention relates to the formulations of bortezomib comprising bortezomib, a non-aqueous solvent or a solubility enhancing agent and a pH modifier or a pH adjusting agent.

In one embodiment, the present invention is a pharmaceutical formulation that includes bortezomib and monothioglycerol.

In another embodiment, the present invention is a pharmaceutical formulation that includes bortezomib and propylene glycol with and without presence of a pH modifying or a pH adjusting agent.

In another embodiment, the present invention relates to the pharmaceutical formulation that comprises bortezomib, propylene glycol and vitamin-E TPGS (d-α-tocopheryl polyethylene glycol 1000 succinate) with and without presence of a pH modifying or a pH adjusting agent.

In another embodiment, present invention is a pharmaceutical formulation that includes bortezomib, propylene glycol and glycerin with and without presence of a pH modifying or a pH adjusting agent.

In another embodiment, present invention relates to the manufacturing process for preparing bortezomib ready-to-dilute composition. The product prepared by following the process of the present invention is stable at room temperature and possesses adequate shelf life, for example for 18-24 months.

### DETAILED DESCRIPTION OF THE INVENTION

Proteosome are the protein complexes in all eukaryotes and archaea. The main function of proteosome is to degrade unnecessary and damaged proteins by proteolysis that breaks chemical bonds.

Proteosome inhibitors are the drugs that block the action of proteosomes, cellular complexes which breakdown proteins. In vitro investigations demonstrated proteosome inhibitor have broad spectrum of anti-proliferative and pro-apoptotic activity against tumors, which makes proteosome inhibitors as potential anti-cancer agents.

The ubiquitin proteosome pathway regulates many processes in the cell which are important for tumor cell growth and survival. Targeting the ubiquitin proteosome pathway has emerged as an effective approach for treatment of human cancer.

Bortezomib is the first proteosome inhibitor anticancer drug approved for treatment of multiple myeloma, relapsed/refractory multiple myeloma and mantle cell lymphoma.

Lactacystin is the natural product which is the first non-peptidic proteosome inhibitor, while disulfiram and epigallocatechin-3-gallate are the other drugs which are proposed to be proteosome inhibitors.

Bortezomib, [(1R)-3-methyl-1-[[(2S)-3-phenyl-2-(pyrazine-2-carbonylamino) propanoyl] amino] butyl] boronic acid. Bortezomib has the following chemical structure:

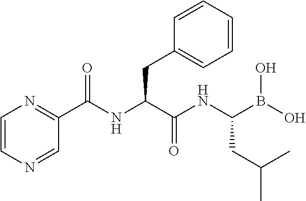 [SMILES: CC(C)C[C@H](NC(=O)[C@H](Cc1ccccc1)NC(=O)c1cnccn1)B(O)O]

The 26S proteosome is a multiprotein complex comprised of 20S core particles and two 19S regulatory particles that degrades ubiquitinated proteins. The 26S proteosome has chymotrypsin-like activity. The boron atom in bortezomib binds to the catalytic site of 26S proteosome with high affinity and specificity. Inhibition of 26S proteosome prevents targeted proteolysis which leads to disruption of multiple signaling cascades within the cell. The disruption of normal homeostatic mechanism can lead to cell death.

Bortezomib is susceptible to oxidative degradation under any experimental conditions. Stress testing and accelerated stability studies revealed that bortezomib is unstable in aqueous solution for injection. Stability of bortezomib slightly improved in mixture of aqueous and non-aqueous solvents.

To improve stability of bortezomib lyophilized and reconstituted prior to injection formulation are designed. Lyophilized formulations are associated with several disadvantages that include time consuming reconstitution process, maintenance of dose precision, limited stability in solution form and also lyophilization process for cytotoxic drugs requires exposure of healthcare professional to cytotoxic vapor of the drugs, so other formulations with selected solvents and solubility enhancing ingredients are required to improve solubility and stability of bortezomib.

The primary object of present invention is to provide ready-to-dilute injectable pharmaceutical formulations of bortezomib comprises of bortezomib, non-aqueous solvents and solubility enhancing agents which impart solubility and stability of active ingredient bortezomib.

Ready-to-dilute injectable formulations of the present invention have following benefits over other liquid formulations:
1) Reconstitution of liquid formulation is not necessary.
2) Extended product stability and stability after dilution.
3) Reduction in manufacturing cost.
4) Simple manufacturing process.

Other aspects of the present invention relate to the pharmaceutical compositions comprising bortezomib for parenteral administration and process of preparation of same. In specific aspects, present invention relates to stable ready-to-dilute injectable pharmaceutical compositions comprising bortezomib with non-aqueous solvents and solubility enhancing ingredients such as nonionic surfactants with pH modifier.

A non-aqueous solvent can be selected from the group consisting of a fixed oil, an alcohol, glycerin, polyethylene glycol, propylene glycol, monothioglycerol, dimethylsulfoxide, ethyl ether and liquid paraffin.

A non-ionic surfactant can be selected from the group consisting of sorbitan esters, polysorbates, poloxamers and vitamin-E TPGS.

As used herein the term “room temperature stable” encompasses stability of the ready-to-use bortezomib injectable composition as disclosed herein at 25±2° C./40±5% RH (relative humidity), 25±2° C./60±5% RH (relative humidity), 25±5° C./40±5% RH (relative humidity), and 25±5° C./60±5% RH (relative humidity) etc. for prolonged time. The term “adequate shelf life” as used herein encompasses stability of the ready-to-use bortezomib injectable composition as disclosed herein at least for 12 months or more, at least 18 months or more or at least 24 months or more.

The term “degradant”, “impurity”, “degradation impurity” and “related substance” as used herein represents the same meaning and can be used interchangeably.

In some of the aspects of the present invention, “stable” or “storage stable”, or “stability” when used with reference to the bortezomib compositions of the present invention or when used “stable bortezomib compositions” or “stability of the bortezomib compositions” all these terms/phrases refer to compositions of the present invention which retain at least about 90%, or about least about 95%, or at least about 96%, or at least about 98%, of the labelled concentration of bortezomib contained in the said composition after storage under typical and/or accelerated conditions. In further aspects, stable bortezomib compositions or stability of the bortezomib compositions refer to less than about 15% (area percent), or less than about 10% (area percent), or less than about 7% (area percent), or less than about 5% (area percent), or less than about 2% (area percent) of bortezomib-related impurities are present after storage under typical and/or accelerated conditions.

The bortezomib compositions of the present invention are stable for prolonged time when stored under storage conditions. The term “storage conditions” as used herein without limitation include typical storage conditions such as 2° C.-8° C., 40° C.±2° C./75±5% RH, 30° C.±2° C./65±5% RH, 25° C.±2° C./40±5% RH, 25° C.±2° C./60±5% RH, and 40° C.±2° C./NMT 25% RH (NMT=not more than) and accelerated conditions such as 40° C.±2° C./75±5% RH.

The term “prolonged time” as used herein indicates that the bortezomib compositions of the present invention are stable for at least 1 month or more, at least 3 months or more, at least 6 months or more or at least 12 months or more when stored under storage conditions.

In some of the aspects, bortezomib compositions of the present invention contain no more than about 15% (area percent), or no more than about 10% (area percent), or no more than about 7% (area percent), or no more than about 5% (area percent), or no more than about 2% (area percent), or no more than about 1% (area percent), or no more than about 0.5% (area percent), or no more than about 0.2% (area percent), or no more than about 0.1% (area percent) any known or unknown single bortezomib-related impurity or other impurity after storage under typical and/or accelerated conditions.

In some of the aspects, bortezomib compositions of the present invention contain no more than about 15% (area percent), or no more than about 10% (area percent), or no more than about 7% (area percent), or no more than about 5% (area percent), or no more than about 2% (area percent), or no more than about 1% (area percent), or no more than about 0.5% (area percent), or no more than about 0.2% (area percent), or no more than about 0.1% (area percent) total bortezomib-related impurities or other impurities after storage under typical and/or accelerated conditions.

Methods for determining the stability of the bortezomib compositions of the present invention with respect to a given parameter are well-known to those of skill in the art. For example, individual impurities and total impurities can be assessed by high-performance liquid chromatography (HPLC) or thin layer chromatography (TLC). Unless otherwise indicated to the contrary, a percentage amount of any individual impurities (known/unknown), or total impurities reported herein in the bortezomib compositions are determined by a peak area percent method using HPLC.

The term “comprise/comprises/comprising” as used herein mean that other ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

The term “about,” as used herein, refers to any value which lies within the range defined by a variation of up to ±10% of the value.

The use of the terms “a” and “an” and “the” and similar referents in the context of describing the invention (especially in the context of the claims) are to be construed to cover both the singular and the plural, unless otherwise indicated herein or clearly contradicted by context.

The term “secondary packaging material” as used herein indicates packaging material made up of any pharmaceutically acceptable & suitable material used to package the bortezomib composition filled vials.

In one embodiment, the present invention discloses a pharmaceutical formulation that includes bortezomib and monothioglycerol. Monothioglycerol serves as an anti-oxidant and protects bortezomib from oxidative degradation. In another embodiment, the present invention discloses a pharmaceutical formulation that includes bortezomib and propylene glycol. The non-aqueous solvent propylene glycol increases the solubility of bortezomib with head space oxygen less than 15% v/v, preferably less than 10% v/v, more preferably less than 5% v/v.

As bortezomib degradation pathway differs in different conditions (media/vehicle, temperature, light etc.) and also observed that impurity trend on stability depends on initial environment and initial and throughout situation control is required. Hence, in the present invention pH optimization trial was also taken.

In another embodiment, the present invention discloses a pharmaceutical formulation comprising bortezomib, propylene glycol and hydrochloric acid to maintain the pH of the formulation between about 1.0 and about 7.0 with head space oxygen less than 15% v/v, preferably less than 10% v/v, more preferably less than 5% v/v and dissolved oxygen less than 10 parts per million (ppm), preferably less than 5 parts per million (ppm), more preferably less than 2 parts per million (ppm).

In another embodiment, the present invention discloses a pharmaceutical formulation that includes bortezomib, propylene glycol, vitamin-E TPGS and hydrochloric acid to maintain pH between about 1.0 and about 7.0 of the formulation. In the said formulation vitamin-E TPGS increases the solubility and stability of the active ingredient, bortezomib. In another embodiment, the present invention discloses a pharmaceutical formulation comprising bortezomib, propylene glycol and glycerin with head space oxygen less than 15% v/v, preferably less than 10% v/v, more preferably less than 5% v/v and dissolved oxygen less than 10 parts per million (ppm), preferably less than 5 parts per million (ppm), more preferably less than 2 parts per million (ppm).

In another embodiment, the present invention discloses a formulation comprising bortezomib, propylene glycol, glycerin and hydrochloric acid to maintain pH of the formulation between about 1.0 and about 7.0 with head space oxygen less than 15% v/v, preferably less than 10% v/v, more preferably less than 5% v/v and dissolved oxygen less than 10 parts per million (ppm), preferably less than 5 parts per million (ppm), more preferably less than 2 parts per million (ppm).

In some of the embodiments, the pH of the bortezomib compositions of the present invention is between about 1.0 and about 7.0. In some of the embodiments, the pH of the bortezomib compositions of the present invention is between about 2.0 and about 6.5. In some of the embodiments, the pH of the bortezomib compositions of the present invention is between about 3.0 and about 5.5. In some of the embodiments, the pH of the bortezomib compositions of the present invention is between about 3.0 and about 6.5. In some of the embodiments, the pH of the bortezomib compositions of the present invention is between about 3.0 and about 7.0. In some of the embodiments, the pH of the bortezomib compositions of the present invention is between about 4.0 and about 6.5. In some of the embodiments, the pH of the bortezomib compositions of the present invention is between about 4.0 and about 7.0.

In some of the aspects, the amount of total impurities present in the bortezomib composition of the present invention is not more than 10.0% w/w when stored for prolonged time under storage conditions. In some of the aspects, the amount of total impurities present in the bortezomib composition of the present invention is not more than 7.0% w/w when stored for prolonged time under storage conditions. In some of the aspects, the amount of total impurities present in the bortezomib composition of the present invention is not more than 5.0% w/w when stored for prolonged time under storage conditions. In some of the aspects, the amount of total impurities present in the bortezomib composition of the present invention is not more than 3.0% w/w when stored for prolonged time under storage conditions.

The ready-to-dilute bortezomib composition of the present invention may be diluted using any suitable pharmaceutically acceptable diluent known to a person skilled in the art. However, preferred diluent is 0.9% sodium chloride. The prescribing information for Velcade® (marketed bortezomib formulation) reveals that the reconstituted material may be stored for up to eight (8) hours in a syringe; however, total storage time for the reconstituted material must not exceed eight (8) hours when exposed to normal indoor lighting. It is surprisingly found that the ready-to-dilute bortezomib composition of the present invention when diluted with suitable pharmaceutically acceptable diluent, preferably 0.9% sodium chloride, it is stable up to 8 hours or more or up to 12 hours or more or up to 24 hours or more or up to 36 hours or more or up to 48 hours or more or up to 60 hours or more or up to 72 hours or more or up to 4 days or more or up to 5 days or more or up to 6 days or more or up to 7 days or more. The diluted bortezomib composition may be kept under protection from light. As used herein the term “protection from light” refers to preventing exposure to visible and ultraviolet light, e.g., electromagnetic radiation having a wavelength from about 10 nm to about 800 nm. Unless the context requires otherwise, the term “light” means UV-Vis light. Typically, visible light ranges from about 400 nm to about 800 nm in wavelength. The terms “protection” and “in the absence of” when referring to exposure to light (e.g., UV-Vis radiation) are used interchangeably herein and mean the amount of light intensity is less than about 50 lux, typically less than about 25 lux, often less than about 10 lux, and most often less than 1 lux.

Suitable pH modifying or pH adjusting agents according to the present invention without limitation include hydrochloric acid, maleic acid, ascorbic acid, oxalic acid, citric acid, carbon dioxide purging and a combination thereof. Preferred pH modifying or pH adjusting agent comprises hydrochloric acid.

Suitable anti-oxidants according to the present invention without limitation include vitamin-E TPGS, monothioglycerol, L-cysteine or a salt thereof, sodium sulfite, L-methionine, ethylenediaminetetraacetic acid or a salt thereof and a combination thereof.

In another embodiment, the present invention discloses the process of manufacturing ready-to-dilute bortezomib injectable formulation comprising bortezomib in the amount from about 7.0 mg/mL to about 17.5 mg/mL, a non-aqueous solvent, a non-buffer pH modifying or pH adjusting agent and optionally an anti-oxidant.

In another embodiment, the present invention discloses a scaling up process for manufacturing ready-to-dilute bortezomib injectable formulation.

The following embodiments describes the manufacturing process for preparing ready-to-dilute bortezomib injectable formulation comprising bortezomib in the amount from about 7.0 mg/mL to about 17.5 mg/mL, a non-aqueous solvent, a non-buffer pH modifying or pH adjusting agent and optionally an anti-oxidant. These embodiments are provided by way of illustration only and should not be construed as to limit the scope and spirit of the claims in any manner.

Batch size: 5 Liter

| Sr. | | Quantity/ |
|---|---|---|
| No. | Ingredients | 0.2 mL |
| 1 | Bortezomib | 3.5 mg* |
| 2 | Propylene glycol | Q.S. to 0.2 mL |
| 3 | Hydrochloric acid | 0.00001 mL |
| | | of 10% v/v |

Primary packaging material components

| | | |
|---|---|---|
| 4 | 5 mL amber 20 mm collar European blow | 01 |
| | back vials | |
| 5 | 20 mm grey bromobutyl omni flex plus coated | 01 |
| | rubber stopper (RTU) (V9048 FM259 FLCO) | |
| 6 | 20 mm light blue flip-off aluminum seals | 01 |

Secondary packaging material components

| | | |
|---|---|---|
| 7 | White folded carton per vial | 01 |

Q.S. = Quantity sufficient

Manufacturing Conditions and Process Components:

| Conditions | Requirement | Obervations |
|---|---|---|
| Dispensing | 25 ± 5° C. | API is light sensitive. Carry |
| of API | | out entire dispensing activity |
| | | under protection from light. |
| Nitrogen | USP NF grade | Required during batch |
| | | manufacturing, transferring, |
| | | filtration and filling. |
| Compounding | 25 ± 5° C. | Stable under room temperature |
| temperature | | 25 ± 5° C. |
| Filtration | PES capsule filter | Compatible for 24 hours at |
| | | room temperature |
| Silicon tube | Platinum cured | Compatible for 24 hours at |
| | silicon tube | room temperature |
| | (for all connection) | |

Pre-Manufacturing Operation, Control and Precautions:
Ensure that the area and equipment are clean before commencing the manufacturing operation.
Ensure that the temperature is maintained between about 20° C. and about 30° C. and relative humidity is maintained not more than about 55% RH throughout manufacturing, filling and sealing process.
Product should be protected from light.
API is light sensitive. Therefore, entre activity should be carried out under protection from light.
Protect the vials from light at each stage of storage/inspection, labeling etc.
Ensure that the filtration/filling/capping/external washer is ready before adding API in compounding vessel. Compounding process should be completed within about 5-7 hours after addition of API. The process from the addition of API to stoppering of filled vials should be completed within about 30 hours.

Manufacturing Process:

Preparation of 10% v/v Hydrochloric Acid Solution:

10% v/v hydrochloric acid solution was prepared by adding 10 mL of 37% hydrochloric acid to 50 mL of water for injection (about 20-30° C.). The solution was cooled to about 20-25° C. Make up the volume to 100 mL with water for injection (about 20-30° C.) and mix well.

### Step-1: Bulk Solution Preparation

### Prerequisites:

Before starting compounding, flush the nitrogen from compounding vessel to filling machine for complete removal of water.
Water contamination should be avoided by water residue from the tank and other utensils.
All compounding process should be done with nitrogen purging.
The batch must be prepared under light protection.
Collect the propylene glycol into the suitable container and weight of the container should be measured before weighing, after dispensing and transferring to manufacturing vessel.
Dispense propylene glycol in four glass containers in A, B, C and D lots as 2 liters, 2 liters, 1 liter and 0.5 liter.
Use of brine water for compounding vessel may be avoided until and unless it is required.
   Process of preparation:
Collect propylene glycol equivalent to approximately 90% of the batch size into the SS316 manufacturing vessel (capacity: not more than 20 liters) and sparge nitrogen under stirring at speed 340±10 rounds per minute (rpm) till the dissolved oxygen level is achieved below 2.0 parts per million (ppm).
Add 0.25 mL of 10% v/v hydrochloric acid solution prepared above and stir the mixture well for about 60 minutes for uniform mixing.
Add dispensed quantity of bortezomib into the manufacturing vessel very slowly with continuous stirring. Rinsing of API polybag/bottle shall be done with approximately 250/300 gm of propylene glycol. Stir well for about 180 minutes or until clear solution is obtained.
Discard the remaining quantity of 10% v/v hydrochloric acid solution and propylene glycol.
After the preparation of bortezomib bulk solution, it should be sent for the filtration.
The bulk solution should be stored under a nitrogen blanket at 25±5° C. in the tightly closed vessel and under protection from light until filtration and filling.

### Step-2: Filtration

### Prerequisites:

Filter the bulk solution only after the filtration and filling line is ready.
The bulk solution should be filtered and filled within about 10-15 hours of completion of the manufacturing process.
Use of filtration tank having capacity more than 2-5 times of the batch size should be avoided and the head space level should be kept low in the filtration tank.
Filtration process should be completed within about 30-50 minutes.

### Filtration:

Filter the bulk solution prepared in step-1 through a sterilized filter Sartopore 3 (0.22 μm, PES, M/s Sartorius) and collect in an SS316L holding vessel (capacity: not more than 2-5 times of the batch size). Store the bulk solution under nitrogen blanketing till taken for filling of the vials.
During the filtration process, the upstream or inlet pressure should not be more than 2.0 bar.

### Step-3: Filling of Vials

### Prerequisites:

Flush all the vials before filling with the bortezomib solution with nitrogen gas under protection from light.
Use needle in needle for filling of the product.
Cover the tubing coming from filtration tank isolator to filling isolator with aluminum foil.
Keep head space level low in buffer bottle during filling process.

### Filling of Vials:

Pre-nitrogen purging in the vial before filling.
Fill target, fill weights as per below table in the 5 mL amber 20 mm collar European blow back vials.

| mL | gm | % |
|---|---|---|
| 0.2 | 0.2078 | 100.00 |

Fill weight

| | | |
|---|---|---|
| Lower (gm) | Target (gm) | Higher (gm) |
| 0.2078 | 0.2182 | 0.2286 |
| Lower (%) | Target (%) | Higher (%) |
| 100.00 | 105.00 | 110.00 |
| mL | gm | % |
| 0.2 | 0.2078 | 100.00 |

Remark:
(1) Density taken 1.039 gm/mL for calculation.
(2) Consider density for fill weight calculation as obtained in in-process bulk analysis.

Step-4: Stoppering of Vials
Protect vials from light.
Stopper the vials with 20 mm grey bromobutyl omni flex plus coated rubber stopper (RTU) (V9048 FM259 FLCO) immediately after the filling.

### Step-5: Capping of Vials

Seal the vial using 20 mm light blue color aluminum flip off seal.
Once filled, protect vials from light.
Perform the head space oxygen analysis on 4 vials at initial, middle and end of vial sealing.
Bortezomib injection 3.5 mg/0.2 mL semi-finished product vials should be transferred to 25±5° C. storage room by protecting vials from light.

### Step-6: Visual Inspection of Vials

Protect the vials from light.
Take only one tray at a time. When 3-4 vials are under visual inspection, keep remaining vials kept in a tray protected from light and outside visual inspection booth.

### Step-7: Packaging

Protect from light and take one tray at a time.
Pack each vial filled with the product into white folded carton.
Store bulk cartons of the drug product at 25±5° C. Protect vials from light.

It is important and advantageous to minimize the contact area between the bortezomib bulk solution and the container or vessel in which it is contained or kept. The container or vessel as used above, without limitation includes compounding vessel, manufacturing vessel, filtration tank, bulk solution holding vessel, reactor etc. made up of any material. Further, when the relatively small amount of bortezomib solution is contained or kept in a vessel or tank or reactor having bigger size or higher capacity (for example more than about 2-5 times of the batch size), it is observed that the amount of total impurities in the bortezomib solution increases dramatically. One of the solutions to this problem is to use such a vessel or tank having less capacity or small size (for example not more than about 2-5 times of the batch size). However, the size of the vessel or tank or reactor depends upon the batch size. For example, when the batch size is 5 liters then the size of the vessel or tank or reactor in which bortezomib solution is contained or kept may be less than 50 liters, preferably less than 20 liters, more preferably less than 10 liters. It is also pertinent to note that it is not only the size of the vessel or tank or reactor which plays an important role. The surface area or diameter of the vessel or tank or reactor which comes into contact with the bortezomib solution also plays an important role and therefore one may be able to decrease the surface area by using the vessel or tank or reactor having high capacity but having small diameter. A skilled person, therefore, may also explore other ways to minimize the contact between the bortezomib solution and the surface area of the vessel or tank or reactor and also the contact period and thereby attempt to minimize the amount of total impurities in the bortezomib composition. Such other ways are also well within the ambit of the present invention.

It is also important and advantageous to minimize the contact area as well as contact time between the filtered bortezomib solution and the surface area of the filtration tank or vessel in which the bortezomib solution is collected after filtration. When the relative small amount of bortezomib solution is collected in a filtration tank or vessel having bigger size or higher capacity (for example more than 2-5 times of the batch size), it is observed that the amount of total impurities in the bortezomib solution increases dramatically.

One of the solutions to above said problem is to filter the bortezomib bulk solution using a filtration tank or vessel having less capacity or small size (for example not more than 2-5 times of the batch size). However, the size of the filtration tank or vessel depends upon the batch size. For example, when the batch size is 5 liters then the size of the filtration tank or vessel may be less than 50 liters, preferably less than 20 liters, more preferably less than 10 liters. It is also pertinent to note that it is not only the size of the filtration tank or vessel which plays an important role. The surface area or diameter of the vessel or filtration tank which comes into contact with the bortezomib solution also plays an important role and therefore one may be able to decrease the surface area by using the filtration tank or vessel having high capacity but having small diameter. A skilled person, therefore, may also explore other ways to minimize the contact between the filtered bortezomib solution and the surface area of the filtration tank or vessel and also the contact period and thereby attempt to minimize the amount of total impurities in the bortezomib composition. Such other ways are also well within the ambit of the present invention.

The capacity of any container or vessel or reactor or utensil or tank being used in the manufacturing process of the bortezomib composition according to the present invention may not be more than 2-5 times of the batch size. In other words, the occupied volume of the container or vessel or reactor or utensil or tank by the batch should not be less than about 90% v/v of the rated volume or should not be less than about 80% v/v of the rated volume or should not be less than about 70% v/v of the rated volume or should not be less than about 60% v/v of the rated volume or should not be less than about 50% v/v of the rated volume or should not be less than about 40% v/v of the rated volume or should not be less than about 30% v/v of the rated volume or should not be less than about 20% v/v of the rated volume or should not be less than about 10% v/v of the rated volume. The term “rated volume” as used herein indicates the capacity of the container or vessel or reactor or utensil or tank used in the manufacturing process. The term “occupied volume” as used herein indicates volume or space of the container occupied by the batch and/or bortezomib bulk solution.

The term “filtrate” as used herein indicates bortezomib solution collected in a filtration tank after filtration. The term “filtration tank” as used herein indicates any vessel made up of any material that is used to collect bortezomib solution after filtration.

The term “vessel”, “tank”, “container”, “utensil” or “reactor” as used herein indicates the container or vessel made up of any suitable material which can be used in the industry and which is used in the entire manufacturing process of ready-to-use bortezomib compositions of the present invention. These terms can be used interchangeably and represents the same meaning. These terms without limitation include compounding vessel, manufacturing vessel, filtration tank, bulk solution holding vessel, reactor etc.

Following data illustrates that the total amount of impurities was increased to more than 10.0% w/w after three months when the bigger size filtration tank or vessel was used for relatively small size batch and the contact between the filtered bortezomib solution and the surface area of the filtration tank or vessel was more.

Batch size: 5 liters

25 ± 2° C./60 ± 5% RH

| Parameters | Initial | 1 month | 3 months |
|---|---|---|---|
| Total impurities | 0.27% w/w | 3.61% w/w | 11.17% w/w |

Further, it is also observed that when the entire process of manufacturing bortezomib solution including filtration and filling & sealing of the vials is completed within about 30 hours or less, preferably within about 24 hours or less, more preferably within about 20 hours or less, most preferably within about 16 hours or less the amount of total impurities in the bortezomib solution is less, preferably not more than 10.0% w/w. But when the total time of entire process exceeds about 30 hours, amount of total impurities dramatically increases in the bortezomib solution. Following data illustrates that the total amount of impurities was increased to more than 10.0% w/w after three months when the total time of above said process was not controlled and was exceeded 30 hours.

Batch size: 5 liters

25 ± 2° C./60 ± 5% RH

| Parameters | Initial | 1 month | 3 months |
|---|---|---|---|
| Total impurities | 0.35% w/w | 2.24% w/w | 10.90% w/w |

Following data illustrates that ready-to-dilute bortezomib injectable composition as disclosed in the present invention when prepared according to the manufacturing process as disclosed herein was found stable at room temperature for prolonged time.

Batch size: 5 liters

25 ± 2° C./60 ± 5% RH

| Parameters | Initial | 1 month | 3 months |
|---|---|---|---|
| Total impurities | 0.19% w/w | 0.27% w/w | 0.71% w/w |

It should be understood that various changes and modifications to the embodiments described herein will be apparent to those skilled in the art. Such changes and modifications can be made without departing from the spirit and scope of the subject matter of the present invention and without diminishing its intended advantages. It is therefore intended that such changes and modifications be covered within the scope of the present invention and appended claims.

What is claimed is:

1. A composition for a ready-to-dilute injectable pharmaceutical formulation of bortezomib comprising:
about 7.0 mg/mL to about 17.5 mg/mL bortezomib;
a non-aqueous solvent;
a non-buffer pH modifying or pH adjusting agent; and
optionally an anti-oxidant,
wherein the amount of dissolved oxygen in said composition is 10 parts per million (ppm) or less.

2. The composition as claimed in claim 1, wherein said non-aqueous solvent is selected from the group consisting of a fixed oil, an alcohol, glycerin, polyethylene glycol, propylene glycol, monothioglycerol, dimethylsulfoxide, ethyl ether, liquid paraffin, and a combination thereof.

3. The composition as claimed in claim 1, wherein said non-buffer pH modifying or pH adjusting agent is selected from the group consisting of hydrochloric acid, maleic acid, ascorbic acid, oxalic acid, carbonic acid, and a combination thereof.

4. The composition as claimed in claim 1, wherein said antioxidant is selected from the group consisting of d-α-tocopheryl polyethylene glycol 1000 succinate (“Vitamin-E TPGS”), monothioglycerol, L-cysteine hydrochloride, sodium sulphite, L-methionine, disodium EDTA, and a combination thereof.

5. The composition as claimed in claim 3, wherein said pH modifying or pH adjusting agent comprises hydrochloric acid.

6. The composition as claimed in claim 1, wherein pH of said composition is between about pH 1.0 and about pH 7.0.

7. The composition as claimed in claim 1, wherein the amount of total impurities present in said composition is not more than about 10.0% w/w.

8. The composition as claimed in claim 1, wherein said composition is stable at room temperature.

9. The composition as claimed in claim 1, wherein said composition upon dilution with pharmaceutically acceptable diluent is stable from about 8 hours up to about 7 days.

10. A non-aqueous solution composition of bortezomib produced by the following process:
removing dissolved oxygen from a non-aqueous solvent to produce a substantially oxygen free non-aqueous solvent;
adding a pH modifying or pH adjusting agent to adjust the pH of said substantially oxygen free non-aqueous solvent to from about pH 1.0 to about pH 7.0;
adding bortezomib to said pH adjusted substantially oxygen free non-aqueous solvent to produce said non-aqueous solution composition, wherein said non-aqueous solution composition comprises from about 7.0 mg/mL to about 17.5 mg/mL of bortezomib; and
optionally adding an anti-oxidant to said non-aqueous solution composition.

11. The non-aqueous solution composition of bortezomib of claim 10, wherein said process is carried out at temperature of about 20° C. to about 30° C. and under relative humidity (RH) of not more than about 55% RH.

12. The non-aqueous solution composition of bortezomib of claim 10, wherein said process is carried out in the absence of visible light.

13. The non-aqueous solution composition of bortezomib of claim 10, wherein said process of removing dissolved oxygen comprises purging said non-aqueous solvent with nitrogen to remove dissolved oxygen.

14. The non-aqueous solution composition of bortezomib of claim 10, wherein said process further comprises a step of filtering said non-aqueous solution composition under nitrogen atmosphere to maintain head space oxygen of not more than 15% v/v.

15. The non-aqueous solution composition of bortezomib of claim 14, wherein said process further comprises a step of filling vials with said filtered non-aqueous solution composition under nitrogen atmosphere in the absence of visible light.

16. The non-aqueous solution composition of bortezomib of claim 10, wherein said process comprises use of a container in which occupied volume of said container by the batch is not less than 20% v/v of the rated volume of said container.

17. The non-aqueous solution composition of bortezomib of claim 10, wherein the dissolved oxygen level maintained throughout the said process is not more than 10 parts per million (ppm).

18. A process for producing a non-aqueous solution composition of bortezomib of claim 1, wherein said process comprises:
removing dissolved oxygen from a non-aqueous solvent to produce a substantially oxygen free non-aqueous solvent;
adding a pH modifying or pH adjusting agent to adjust the pH of said substantially oxygen free non-aqueous solvent to from about pH 1.0 to about pH 7.0;
adding bortezomib to said pH adjusted substantially oxygen free non-aqueous solvent to produce said non-aqueous solution composition of claim 1; and
optionally adding an anti-oxidant to said non-aqueous solution composition.

19. The process of claim 18, wherein the dissolved oxygen level maintained throughout the said process is not more than 10 parts per million (ppm).

20. The process of claim 18, wherein said dissolved oxygen is removed from said non-aqueous solvent by purging with nitrogen gas.
